(19) United States
(12) Patent Application Publication
(10) Pub. No.: US 2009/0004718 A1
(43) Pub. Date: Jan. 1, 2009

(54) Antioxidant Fermenting Microorganism Agent Reducing Volatile Organic Compounds From Polyurethane Foam, and Urethane Foam Comprising the Same

(75) Inventors: Taek Lim, Gyeonggi-do (KR); Jae-Yong Ko, Gyeonggi-do (KR); Eun-Ju Lee, Seoul (KR); In-Sung Kim, Gyeonggi-do (KR); Chang-Hong Lee, Jeju-do (KR)

Correspondence Address: HAMRE, SCHUMANN, MUELLER & LARSON, P.C., P.O. BOX 2902, MINNEAPOLIS, MN, 55402-0902, US

(73) Assignees: DYMOS INC., Seosan City, Chungnam (KR); SEOUL NATIONAL UNIVERSITY INDUSTRY FOUNDATION, GWANAK-GU SEOUL (KR)

(21) Appl. No.: 12/224,154

(22) PCT Filed: May 9, 2007
(86) PCT No.: PCT/KR2007/002268

§ 371 Date: Aug. 18, 2008

(30) Foreign Application Priority Data
May 10, 2006 (KR) 10-2006-0042034

(51) Int. Cl. C12N 11/04; C12N 1/20

(52) U.S. Cl. 435/182; 435/252.4

(57) ABSTRACT

Disclosed is an antioxidant fermenting microorganism agent and a polyurethane foam including the same. The antioxidant fermenting microorganism agent is used to generate a substance that is capable of reducing an amount of volatile organic compounds generated from the polyurethane foam and has an antimicrobial ability. The polyurethane foam containing the antioxidant fermenting microorganism according to the present invention can be used as the material for the interior of a vehicle to protect humans from noxious substances causing sick car syndrome.

### TECHNICAL FIELD

The present invention relates to an antioxidant fermenting microorganism agent that is used to reduce a volatile organic compound generated from a polyurethane foam used as the material for the interior of a vehicle, and a polyurethane foam including the same.

### BACKGROUND ART

A polyurethane foam that is used as the material for the interior of a vehicle is applied to various types of goods in order to block heat and absorb impact in the vehicle. The polyurethane foam is made from a 100% chemical agent, thereby emitting various types of volatile organic compounds if heat is applied to polyurethane foam products, and the volatile organic compounds negatively affect humans.

In respect to noxiousness of many volatile organic compounds that causes a sick house syndrome, a sick car syndrome, and the like, there are various types of regulations all over the world. However, imperfect effort has been made to examine specific symptoms caused by the volatile organic compounds and to reduce the volatile organic compounds.

Recently, photocatalysts and various types of chemical substances have been used to reduce volatile organic compounds generated from the polyurethane foams that are used as the material for the interior of a vehicle, but are not selected as a critical technology because of a very high treatment cost.

### DISCLOSURE OF INVENTION

### Technical Problem

It is an object of the present invention to provide an antioxidant fermenting microorganism agent that is used to radically reduce a volatile organic compound generated from a polyurethane foam used as the material for the interior of a vehicle at low cost, and a polyurethane foam including the same.

### Technical Solution

The present invention provides an antioxidant fermenting microorganism agent to reduce a volatile organic compound generated from a polyurethane foam, wherein said agent includes a microorganism mixture, water, an organic carbon source, and a natural salt at a weight ratio of 3 to 7:50 to 150:3 to 7:0.5 to 1.5 and wherein the microorganism mixture contains photosynthetic bacteria, yeast, actinomyces, and lactobacillus at a germ number ratio of 50 to 150:0.5 to 1.5:0.5 to 1.5:3 to 7.

Furthermore, the present invention provides a polyurethane foam containing the antioxidant fermenting microorganism agent.

Furthermore, the present invention provides a seat for vehicles made of the polyurethane foam.

### ADVANTAGEOUS EFFECTS

An antioxidant fermenting microorganism agent according to the present invention is added during the processing of a polyurethane foam to radically remove the cause of a volatile organic compound, thereby reducing the amount of the volatile organic compound generated from the processed polyurethane foam. In addition, the antioxidant fermenting microorganism agent generates an antimicrobial substance such as a benzoic acid to induce an antimicrobial action on the polyurethane foam. Furthermore, the antioxidant fermenting microorganism agent acts on the wastewater generated from a process of producing the polyurethane foam, to biodegrade various types of compounds contained in the wastewater, thus increasing treatment efficiency the wastewater.

### BRIEF DESCRIPTION OF THE DRAWINGS

FIG. 1 is a GC-MS graph of a known polyurethane foam that does not contain an antioxidant fermenting microorganism agent; and

FIG. 2 is a GC-MS graph of a polyurethane foam that contains an antioxidant fermenting microorganism agent.

### BEST MODE FOR CARRYING OUT THE INVENTION

The present invention will be described in detail hereinafter.

The term “An antioxidant fermenting microorganism” in the present invention means a combination of effective microorganism groups that coexist and live symbiotically and are capable of creating an antioxidant substance such as inositol, ubiquinone, and polyphenol using an organic carbon source and natural salts as raw materials through a fermentation process.

The antioxidant fermenting microorganism agent of the present invention contains various types of microorganism species including photosynthetic bacteria, yeast, actinomyces, lactobacillus, and the like, and is environmentally safe material which has been used in fermenting traditional foods. The antioxidant fermenting microorganism agent according to the present invention is added during a process of producing a polyurethane foam to prevent various types of chemical agents from remaining and to decompose the remaining chemical agents, thereby reducing the amount of volatile organic compounds that are generated from the produced polyurethane foam.

The antioxidant fermenting microorganism agent according to the present invention contains a microorganism mixture including photosynthetic bacteria, yeast, actinomyces, and lactobacillus that are mixed with each other at a germ number ratio of 50 to 150:0.5 to 1.5:0.5 to 1.5:3 to 7 to reduce the amount of volatile organic compounds. It is most preferable that the mixing ratio of photosynthetic bacteria, yeast, actinomyces, and lactobacillus be 100:1:1:5.

Examples of photosynthetic bacteria which are contained in the antioxidant fermenting microorganism agent according to the present invention include Rhodopseudomonas palustris, examples of yeast include Saccharomyces cerevisiae, examples of actinomyces include Streptomyces albus, and examples of lactobacillus include Lactobacillus plantarum.

The above-mentioned germs that act as the matrices are appropriately mixed with each other, and water, organic carbon sources, and natural salts are added at a weight ratio of 50 to 150:3 to 7:0.5 to 1.5 in a fermentation culture fluid form, and fermentation is completely performed in an anaerobic state at 20 to 30° C. for 7 to 10 days to produce the antioxidant fermenting microorganism according to the present invention. Preferably, the fermentation culture fluid contains water, organic carbon sources, and natural salts at a weight ratio of 100:5:1.

The organic carbon source is a typical carbon nutritive element and preferable examples thereof include syrup, and preferable examples of the natural salts include deep-sea natural salts that are not contaminated.

Uncontaminated natural sea salts are fermented with the microorganism agent according to the present invention for 7 days to produce the natural salts which are used in the present invention.

In addition, the present invention provides polyurethane foam that contains the antioxidant fermenting microorganism agent.

The polyurethane foam is produced by a crosslinking reaction and foaming of polyol and isocyanate, and the antioxidant fermenting microorganism agent according to the present invention is preferably mixed with polyol before crosslinking.

In respect to a process of adding the antioxidant fermenting microorganism agent to polyol, the addition may be performed while making polyol or may be performed in a preparation tank for foaming of polyurethane. Specifically, the polyurethane foam contains 0.01 to 0.02 wt % of antioxidant fermenting microorganism agent based on the amount of added polyol.

In this connection, in the case of when the content of the antioxidant fermenting microorganism agent is less than 0.01 wt % of polyol, removal efficiency of odors of the volatile organic compounds is reduced. In the case of when the content is more than 0.02 wt %, there is a problem in that other odors different from those of the volatile organic compounds are generated due to the fermentation of the microorganism agent.

Furthermore, the antioxidant fermenting microorganism agent according to the present invention is applied on the entire surface of the polyurethane foam and the sheet product by using a spray process to form a thin film. In the case of when the resulting structure is left in an open space for 24 hours, source substances of odor and the volatile organic compounds may be decomposed and removed.

As described above, in the present invention, the antioxidant fermenting microorganism agent is added during the production process to produce the polyurethane foam or sprayed on the produced polyurethane foam, thereby reducing radical factors that are noxious to humans such as odor and volatile organic compounds and the like.

Once the polyurethane foam according to the present invention is used, the sick car syndrome that is caused by vehicles may be reduced.

In addition, the antioxidant fermenting microorganism according to the present invention generates secondary products that act as antimicrobial action such as benzoic acids and antioxidant substances including inositol, ubiquinone, and polyphenols by fermentation process so that the anaerobic microorganisms are capable of enduring oxygen free radicals. The secondary products may suppress multiplication of noxious mold and bacteria that are generated in the polyurethane foam on the inside of the vehicle in a humid environment.

### Mode for the Invention

Hereinafter, the present invention will be described in detail in light of Examples and Experimental Examples. The present invention may, however, be embodied in many different forms and should not be construed as being limited to the Examples and Experimental Examples set forth herein. Rather, these Examples and Experimental Examples are provided such that this disclosure will be thorough and complete and will fully convey the concept of the present invention to those skilled in the art.

### EXAMPLE 1

### Antioxidant Fermenting Microorganism Agent

Rhodopseudomonas palustris that was photosynthetic bacteria, Saccharomyces cerevisiae that was yeast, Streptomyces albus that was actinomyces, and Lactobacillus plantarum that was lactobacillus were mixed with each other at a ratio of 100:1:1:5. The microorganism mixture, water, the organic carbon source, and the natural salts were mixed with each other at a ratio of 5:100:5:1 and completely fermented in an anaerobic state at 20 to 30° C. for 7 to 10 days, and the resulting substance was used to reduce the odor. The above-mentioned germs were isolated and cultured in a natural state.

### EXAMPLE 2

Materials that were typically used to produce the polyurethane foam were prepared. In this connection, polyol and 0.01 wt % of the antioxidant fermenting microorganism agent based on the amount of polyol were added, agitated in a tank for 1 to 2 hours, and subjected to the crosslinking reaction with isocyanate and foaming to produce a polyurethane foam which is capable of reducing the generation of volatile organic compounds (VOCs) therefrom. The crosslinking reaction and the foaming according to the present example were performed through the same procedure as a known process, and all experiments were performed at a normal temperature (18 to 25° C.).

### EXPERIMENTAL EXAMPLE

The untreated polyurethane foam that was produced by using a typical process (the polyurethane foam that did not contain antioxidant fermenting microorganism agent according to the present invention, weight: 3.4 g) and the treated polyurethane foam that was produced in Example 2 (weight: 3.4 g) were cut into rectangular pieces and the pieces were used to perform an odor analysis test.

The polyurethane foams were put separately into the chambers for generating the volatile organic compounds (5 l Pyrex chamber) and the chambers were completely sealed. The chambers for generating the volatile organic compounds in which the polyurethane foams were contained were treated in a thermostat at 80° C. for 12 hours. The volatile organic compounds that were generated from the chambers that were treated at the constant temperature of 80° C. were adsorbed onto an adsorption tube (Tenax TA Thermal Desorption tube).

The adsorption process included creating a vacuum after providing a moisture and air removing filter to an inlet of each of the chambers, providing the adsorption tube to an outlet of each of the chambers, and creating a vacuum by using a vacuum pump (12 to 15 □/min) for 1 min.

In the case of when a vacuum was created by using the vacuum pump (12 to 15 □/min) for 1 min, since each of the chambers for generating the volatile organic compounds was produced to have the volume of 5 l, the generated volatile organic compounds were adsorbed onto media in the adsorption tube while all gases emitted from the chambers passed through the adsorption tube (the adsorption tube was treated at 380° C. for 3 hours before being used to maintain the desired active state).

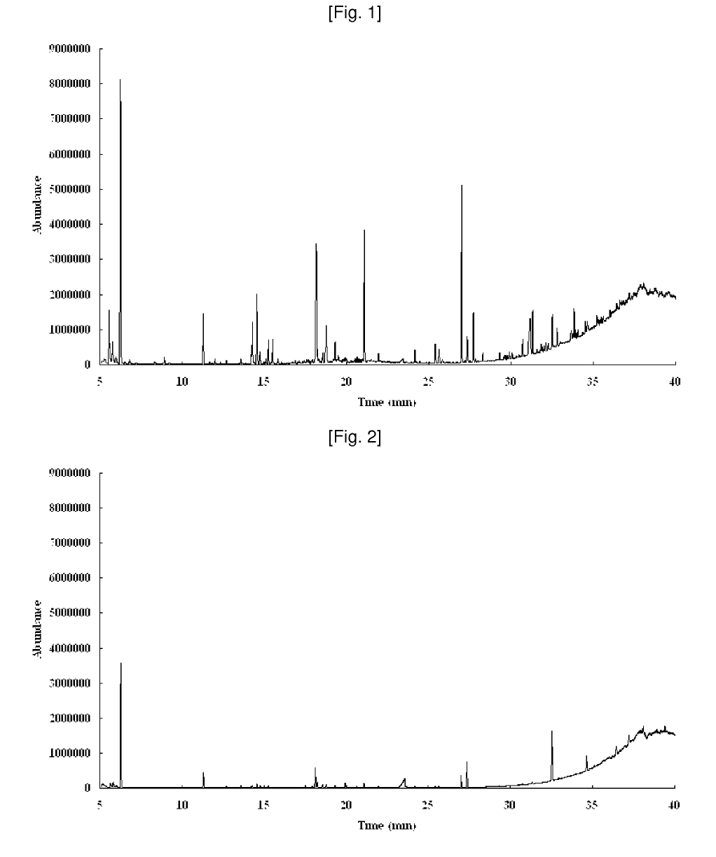

The adsorption tube was connected to GC-MS to perform qualitative analysis for 40 min, a library search was performed to confirm the components, and the results of the meaningful volatile organic compounds are described in the following Table 1. FIGS. 1 and 2 are GC-mass graphs of the untreated polyurethane foam and the treated polyurethane foam, respectively.

TABLE 1

| | | | | Increase | |
|---|---|---|---|---|---|
| | | Generation amount | | and decrease | |
| | | (□/g) | | (%) | |
| No. | Component | Untreated | Treated | Increase | Decrease |
| 1 | Ethanol | 1.15 | 0 | — | 100 |
| 2 | Acetone | 0.50 | 0 | — | 100 |
| 3 | Carbon disulfide | 7.15 | 2.51 | — | 64.9 |
| 4 | Toluene | 1.00 | 0.27 | — | 73.0 |
| 5 | Methyllaurate | 0.96 | 0 | — | 100 |
| 6 | 1,4-dimethyl-benzene | 1.71 | 0.10 | — | 94.2 |
| 7 | 1,2-dimethyl-benzene | 0.49 | 0 | — | 100 |
| 8 | Octamethyl-trisiloxane | 0.42 | 0 | — | 100 |
| 9 | 2,2′-azobis[2-methyl]- | 3.33 | 0 | — | 100 |
| | propanenitrile | | | | |
| 10 | 2,6,6-trimethyl- | 0 | 0.11 | 100 | — |
| | bicyclo[3,1,1]heptane | | | | |
| 11 | Octamethyl-cyclotetra- | 0.89 | 0 | — | 100 |
| | siloxane | | | | |
| 12 | 1,2-dichloro-benzene | 0.46 | 0 | — | 100 |
| 13 | Decamethyl-tetra- | 2.46 | 0 | — | 100 |
| | siloxane | | | | |
| 14 | Benzoic acid | 0 | 0.68 | 100 | — |
| 15 | Benzothiazole | 0.46 | 0.00 | — | 100 |
| 16 | Dodecamethyl-penta- | 3.06 | 0.20 | — | 93.5 |
| | siloxane | | | | |
| 17 | Phthalic anhydride | 0.68 | 0.64 | — | 5.9 |
| 18 | 2-amino-Benzenecar- | 1.14 | 0 | — | 100 |
| | bothioamide | | | | |
| 19 | Phenylmaleic | 0 | 1.21 | 100 | — |
| | anhydride | | | | |
| 20 | Diphenyl-methanone | 0 | 0.33 | 100 | — |

| | | |
|---|---|---|
| TVOCs | — | 74.8 |

As shown in the above Table 1, the generation amount of total volatile organic substances (TVOC) of the treated sample was 25.2 which were reduced by 74.8% based on 100 of the untreated urethane foam. Specifically, the amount of carbon disulfide which is noxious to humans and has an odor was reduced by 64.9% from 7.15 □/g to 2.51 □n/g.

Furthermore, the amount of toluene was reduced by 73% from 1.00 □/g to 0.27 □/g. Chemical substances having odors such as acetone, decamethyl-tetrasiloxane, and 2-amino-benzenecarbothioamide were not detected in the treated polyurethane foam. The content of benzoic acid that was not detected in the untreated polyurethane foam was increased to 0.68 □/g in the treated polyurethane foam. The benzoic acid was the antimicrobial substance, thus it can be seen that the antioxidant fermenting microorganism agent according to the present invention has an antimicrobial ability.

1. An antioxidant fermenting microorganism agent comprising:
a microorganism mixture that contains photosynthetic bacteria, yeast, actinomyces, and lactobacillus at a germ number ratio of 50 to 150:0.5 to 1.5:0.5 to 1.5:3 to 7;
water;
an organic carbon source; and
a natural salt,
wherein the microorganism mixture, the water, the organic carbon source, and the natural salt are mixed at a weight ratio of 3 to 7:50 to 150:3 to 7:0.5 to 1.5 to reduce a volatile organic compound generated from a polyurethane foam.

2. The antioxidant fermenting microorganism agent as set forth in claim 1, wherein the microorganism mixture contains photosynthetic bacteria, yeast, actinomyces, and lactobacillus mixed with each other at a weight ratio of 100:1:1:5.

3. The antioxidant fermenting microorganism agent as set forth in claim 1, wherein the microorganism mixture, the water, the organic carbon source, and the natural salt are mixed at a weight ratio of 5:100:5:1.

4. A polyurethane foam comprising the antioxidant fermenting microorganism agent according to claim 1.

5. A polyurethane foam comprising the antioxidant fermenting microorganism agent according to claim 2.

6. A polyurethane foam comprising the antioxidant fermenting microorganism agent according to claim 3.

7. The polyurethane foam as set forth in claim 4, wherein the antioxidant fermenting microorganism agent is contained in the polyurethane foam in an amount of 0.01 to 0.02 wt % based on a total amount of polyol that constitutes the polyurethane foam.

8. The polyurethane foam as set forth in claim 5, wherein the antioxidant fermenting microorganism agent is contained in the polyurethane foam in an amount of 0.01 to 0.02 wt % based on a total amount of polyol that constitutes the polyurethane foam.

9. The polyurethane foam as set forth in claim 6, wherein the antioxidant fermenting microorganism agent is contained in the polyurethane foam in an amount of 0.01 to 0.02 wt % based on a total amount of polyol that constitutes the polyurethane foam.

10. A sheet for vehicles which is produced by using the polyurethane foam according to claim 4.

11. A sheet for vehicles which is produced by using the polyurethane foam according to claim 5.

12. A sheet for vehicles which is produced by using the polyurethane foam according to claim 6.
